(19) United States
(12) Patent Application Publication
(10) Pub. No.: US 2026/0306022 A1
(43) Pub. Date: Oct. 8, 2026

(54) WEARABLE SENSOR-BASED MULTI-DISEASE DETECTION CONTINUAL LEARNING FRAMEWORK

(71) Applicant: THE TRUSTEES OF PRINCETON UNIVERSITY, Princeton, NJ (US)

(72) Inventors: Chia-Hao Li, Plainsboro, NJ (US); Niraj Li, PRINCETON, NJ (US)

(73) Assignee: THE TRUSTEES OF PRINCETON UNIVERSITY, Princeton, NJ (US)

(21) Appl. No.: 19/479,927

(22) PCT Filed: May 2, 2024
(86) PCT No.: PCT/US2024/027370

§ 371 Date: Oct. 29, 2025

Related U.S. Application Data
(60) Provisional application No. 63/464,235, filed on May 5, 2023.

(51) Int. Cl. A61B 5/00

(52) U.S. Cl. CPC A61B 5/681; A61B 5/7267

(57) ABSTRACT

Disclosed is a non-transitory computer-readable storage device containing instructions that, when executed by one or more processing units, causes the one or more processing units to deploy a multi-headed deep neural network (DNN) model that includes an exemplar-replay-style continuous learning (CL) algorithm. The DNN model may be configured to receive information originating from a wearable medical sensor and receive replay data from the CL algorithm. The CL algorithm may be configured with a data preservation module and a synthetic data generation module. The data preservation module may be configured to preserve a subset of training data from one or more previous missions based on an average training loss of each data instance. The synthetic data generation module may be configured to model a probability distribution of real training data and then generate synthetic data sufficient for replays while maintaining data privacy.

### CROSS-REFERENCE TO RELATED APPLICATIONS

The present application claims priority to U.S. Provisional Patent Application No. 63/464,235, filed May 5, 2023, the contents of which are incorporated by reference herein in its entirety.

### STATEMENT REGARDING FEDERALLY SPONSORED RESEARCH OR DEVELOPMENT

This invention was made with government support under Grant No. CNS-1907381 awarded by the National Science Foundation. The government has certain rights in the invention.

### TECHNICAL FIELD

The present disclosure is drawn to medical diagnostic systems, and in particular, diagnostic systems utilizing continuous learning techniques with wearable medical sensor (WMS) data.

### BACKGROUND

Physical illnesses and mental health problems impact the well-being of billions of people around the globe. For centuries, disease detection has been a stressful and time-consuming process for patients. Patients have to visit a physician and undergo a series of physical and even invasive examinations. Fortunately, the emergence of wearable medical sensors (WMSs) and modern advances in artificial intelligence (AI) and machine learning (ML) point to a promising approach to address these problems. WMSs enable continuous monitoring of physiological signals in a passive, user-transparent, and non-invasive manner. A well-designed and well-trained ML model can then analyze the physiological signals captured by WMSs and perform efficient and effective disease detection. This can now be enabled even in an out-of-clinic scenario.

However, conventional ML-driven disease detection methods rely on customizing an ML model for each disease based on its associated WMS data. They lack adaptability to changes in data distributions and the addition of new classification classes for a given disease. For instance, the ML model trained with physiological signals collected from the elderly might not accurately detect the disease in young adults. The model trained with data from healthy individuals and symptomatic COVID-19 patients might not be able to detect the virus in asymptomatic patients. In conventional ML-driven detection methods, the model needs to be retrained with new data to adapt to data domain shifts or new classification classes. Even then, the model may overfit on new data from different domains or classes and suffer from performance deterioration on previously trained missions due to a concept called catastrophic forgetting. Moreover, the model trained for detecting one disease cannot be used to detect others. Therefore, new models need to be designed and trained from scratch to detect new diseases. It makes conventional ML-driven disease detection methods suboptimal since placing multiple disease-detecting models on smartwatches or smart-phones (where WMSs reside) increases memory footprint and drains the battery much faster.

### BRIEF SUMMARY

In various aspects, a non-transitory computer-readable storage device may be provided. The device may contain instructions that, when executed by one or more processing units, causes the one or more processing units to be configured to, either individually or collectively, deploy a multi-headed deep neural network (DNN) model that includes an exemplar-replay-style continuous learning (CL) algorithm. The DNN model may be configured to receive information originating from a wearable medical sensor and receive replay data from the CL algorithm. The CL algorithm may be configured with a data preservation module and a synthetic data generation module. The data preservation module may be configured to preserve a subset of training data from one or more previous missions based on an average training loss of each data instance. The synthetic data generation module may be configured to model a probability distribution of real training data and then generate synthetic data sufficient for replays while maintaining data privacy.

A model size of the multi-headed DNN model may be less than 1 MB, or less than 500 KB. A model size of the DNN model may increase by less than 2%, or less than 1%, for each additional detection head that is added to the multi-headed DNN model.

The synthetic data generation module may be configured to model a joint multivariate probability distributions of real training data through both parametric and non-parametric density estimation methods, and generate synthetic data based on the learned probability distribution.

In various aspects, a system for continually learning to detect new diseases with a single neural network model may be provided. The system may include a wearable medical sensor. The system may include a non-transitory computer-readable storage device as disclosed herein. The system may include one or more processing units operably coupled to the wearable medical sensor and to the non-transitory computer-readable storage device.

In various aspects, a method for continually learning to detect new diseases with a single neural network model may be provided. The method may include generating data for a current mission by receiving data from a wearable medical sensor and preprocessing the received data. The method may include receiving data from previous missions from a exemplar-replay-style continuous learning (CL) algorithm. The method may include analyzing the data for the current mission to determine if more output neurons and/or detection heads are needed in a multi-headed deep neural network (DNN) model for the current mission. If so, the method may include expanding the DNN model include more output neurons and/or detection heads. The method may include training the DNN model jointly with substantially equal data from the current mission and previous missions. The method may include preserving or generating data in one of two fashions. One approach may be to obtaining the average training loss of each data instance in the current mission after training, computing a threshold value for data preservation, and preserving data instances whose average training loss values are above the threshold value for future replays. A second approach may be to model a joint multivariate probability distributions of real training data through both parametric and non-parametric density estimation methods, and sampling as much synthetic data as required from the joint multivariate probability distributions.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic of prior machine learning approaches to detecting multiple diseases.

FIG. 2 is a schematic of a continual learning approach to detecting multiple diseases.

FIG. 3 is a schematic of a system.

FIG. 4 is a schematic of a continual learning framework.

FIGS. 5A-5C are schematic representations of model expansion procedures for multi-disease detection in the domain-incremental scenario (5A), class-incremental scenario (5B), and task-incremental scenario (5C) in DOCTOR.

FIG. 6 is a schematic representation of the DOCTOR framework at test time.

FIG. 7 is a graph showing the training loss of each training data instance in the CovidDeep dataset across 150 epochs.

FIGS. 8-11 are algorithms for data preservation (8), synthetic data generation (9), gaussian mixture model estimation (10), and kernel density estimation (11).

FIG. 12 is a schematic representation of the DNN architecture in DOCTOR, where Dᵢ represents the output head of the i-th disease detection task.

FIG. 13 is a table (Table I) showing domain-incremental CL experimental results.

FIG. 14 is a table (Table II) showing class-incremental CL experimental results.

FIG. 15 is a table (Table III) showing task-incremental CL experimental results.

FIG. 16 is a schematic representation of DOCTOR's simultaneous multi-disease detection application at test time, where C represents the CovidDeep detection head, D symbolizes the DiabDeep detection head, and MH stands for the MHDeep detection head.

FIG. 17 is a table (Table IV) showing experimental results of the ablation study on the data preservation CL algorithm.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

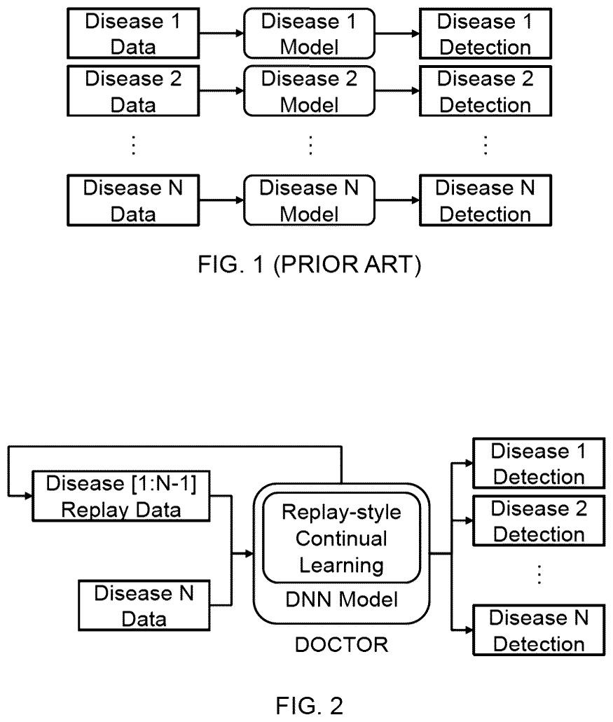

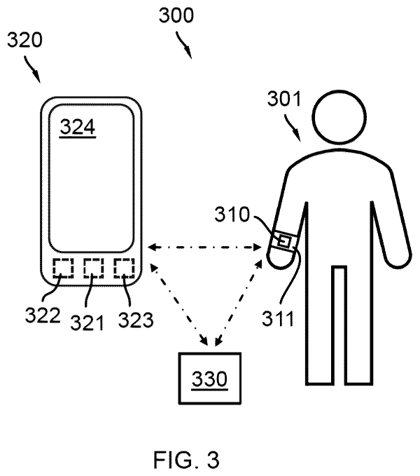

Disclosed is a multi-disease detection continual learning framework based on wearable medical sensors (hereafter “DOCTOR”). FIG. 2 gives an overview of the framework, which can be compared to conventional ML-driven disease detection methods, shown in FIG. 1.

As seen in FIG. 2, DOCTOR performs multi-disease detection with a deep neural network (DNN) and a replay-style continuous learning (CL) algorithm. The CL algorithm enables the framework to continually learn new missions with a single DNN where different data distributions, classification classes, and disease detection tasks are introduced sequentially. In addition, DOCTOR does not require manual feature extraction and operates directly on tabular data collected from commercially available WMSs. It counteracts catastrophic forgetting in the tabular data domain by training the DNN in an exemplar-replay fashion.

The proposed replay-style CL algorithm employs a data preservation method and a synthetic data generation module. The data preservation method preserves subsets of training data from previous missions without incurring excessive computational costs. The preserved training data are sampled from the most informative subsets based on their average training loss values. This approach enables efficient exemplar sampling when computational resources are limited and the preservation of real data is allowed. The synthetic data generation module recreates the joint multivariate probability distributions of the real training data from previous missions. Both parametric and non-parametric density estimation methods may be adopted to model the probability distributions. Then, the module generates synthetic data from the learned probability distributions of previous missions for replays during training. Hence, the synthetic data generation module can generate as much training data as needed when the preservation of real data is infeasible due to memory restrictions or data privacy issues. Moreover, it reduces memory footprint by storing only the learned probability distribution rather than the generated synthetic data.

A multi-head DNN architecture was adopted into the framework. When DOCTOR learns how to make predictions on a new disease detection task, it first creates a new detection head in parallel to those trained for previous tasks. Then, it learns an individual classification probability distribution for the new task at the new head. Meanwhile, the other heads for the previous tasks are fine-tuned through exemplar replays. Due to the individual probability distribution learned for each detection head, the multi-head architecture allows DOCTOR to detect multiple diseases simultaneously based on user WMS data.

Modern developments in ML enable efficient and effective ML-driven disease detection systems. For example, Covid-Deep and MHDeep adopt grow-and-prune algorithms to synthesize optimal DNN architectures to detect the SARS-CoV-2 virus/COVID-19 disease and mental health disorders based on physiological signals collected from commercially available WMSs and smartphones. Similarly, DiabDeep uses grow-and-prune synthesis to deliver DNN models with sparsely-connected layers or sparsely-recurrent layers for diabetes detection based on WMS data and patient demographic information. Miao and Miao developed a DNN model to perform fetal health assessment in complications of pregnancy based on multi-class morphological pattern predictions with cardiotocography data. Ghazal trained a DNN to predict early-stage liver disease with tabular patient data. Dritsas and Trigka used a rotation forest model to predict chronic kidney disease with patient physiological data. Allugunti employed a convolutional neural network (CNN) to classify different types of melanoma skin diseases using patient skin images. Finally, Melekoodappattu combined a customized CNN with image texture attribute extraction to perform breast cancer detection in mammograms. Such approaches are best understood with reference to FIG. 1—data was used to create a specific model for that disease, and that trained model is then used to detect the disease.

Many CL algorithms have been proposed in the literature to counteract the catastrophic forgetting phenomenon in ML models. Recent CL algorithms can mainly be categorized into three groups: regularization-based, architecture-based, and replay-based.

Regularization-based methods use restrictions to limit the update process of model parameters. For example, elastic weight compensation (EWC) and synaptic intelligence (SI) techniques have been used to add regularization penalties to loss functions to mitigate the update process for the weights that are most important for solving previous missions. Li's Learning without Forgetting (LwF) algorithm generates pseudo labels for new data before learning a new mission and uses them to regularize the model to preserve knowledge learned from previous missions. Regularization-based methods address catastrophic forgetting without storing exemplars and incurring additional memory costs. However, they do not achieve satisfactory performance under challenging settings or complex datasets.

Architecture-based methods dynamically expand the model architecture to accommodate new missions. For instance, dynamic expandable networks (DEN) techniques dynamically expand the network capacity to compose a compact, overlapping, and knowledge-sharing structure to learn new missions. SpaceNet trains sparse DNNs in an adaptive way from scratch to produce sparse representations and compress the sparse connections of each task in a compact number of neurons in the class-incremental CL scenario. DualNet constructs a fast-learning system for supervised learning of pattern-separated representation from specific tasks and a slow learning system for unsupervised representation learning of task-agnostic general representation. Architecture-based methods also perform CL without preserving data from past missions. Nevertheless, they require a substantial number of additional parameters and may not scale to a large number of missions.

Replay-based methods sample exemplars or generate synthetic data from previous missions, store them in a data buffer, and replay them with data from new missions to alleviate catastrophic forgetting. For example, iCaRL preserves a representative set of exemplars from previous missions based on herding and replays them with new data to perform nearest-class-mean classification. Similarly, gradient episodic memory (GEM) techniques store previous mission exemplars and replays them with new data to update model parameters in a way that incurs a minimal loss for previous tasks. In addition, Hayes' replay using memory indexing (REMIND) approach implements hippocampal indexing theory with tensor quantization to efficiently store compressed representations for future replays instead of raw images. On the other hand, deep generative replay uses a generative adversarial network to generate synthetic images for previous missions to replay when learning a new mission. Generally, replay-based methods might suffer performance deterioration when buffer size is limited. Moreover, preserving actual data might be infeasible in real-world applications due to data privacy requirements. However, empirically, they achieve the best performance trade-offs compared to the other two methods, even under complex CL scenarios.

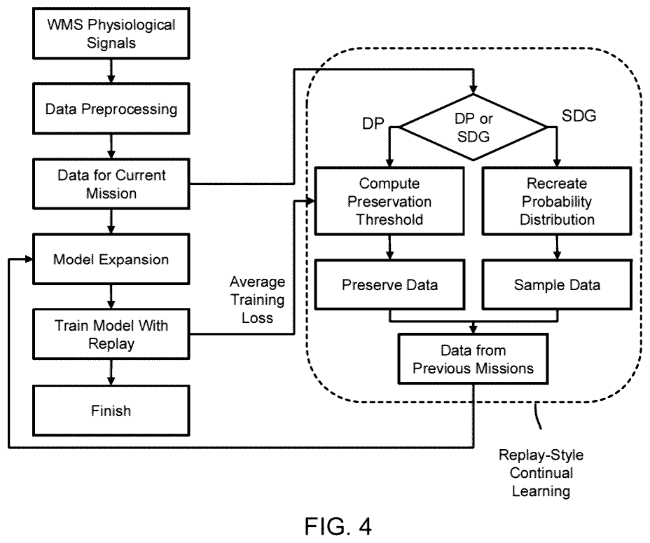

In various aspects, a system for continually learning to detect new diseases with a single neural network model may be provided. Referring to FIG. 3, the system (300) may include a wearable medical sensor (310). The sensor may be present as a standalone device, or as part of multi-purpose device. The sensor may be attached to a user (301) or worn, e.g., using a band (311), a clip, adhesive, or any other appropriate manner.

The system may include one or more processing units (321) operably coupled to the wearable medical sensor. As used herein, the term “processing unit” generally refers to a computational device capable of accepting data and performing mathematical and logical operations as instructed by program instructions. This may include any central processing unit (CPU), graphics processing unit (GPU), core, hardware thread, or other processing construct known or later developed. The term “thread” is used herein to refer to any software or processing unit or arrangement thereof that is configured to support the concurrent execution of multiple operations.

In certain aspects, the processing unit(s) may be present on the wearable sensor (not shown). In a preferred embodiment, the processing unit(s) may be present on a remote device (320), such as a smart phone, tablet, etc. In some embodiments, the processing unit(s) may be present on a cloud-based server (330). In some embodiments, the processing unit(s) may be at a plurality of remote locations (such as on a smart phone and on a cloud-based server). In a preferred embodiment, all the processing unit(s) are present on a single device (such as only on a smart phone).

The system may include a memory (322) operably coupled to the processing unit(s).

The system may include a non-transitory computer-readable storage medium (323) operably coupled to the one or more processing units. The non-transitory computer-readable storage medium may contain instructions. The instructions, when executed by the processing unit(s), may cause the processing unit(s) to, either individually or collectively, deploy a multi-headed deep neural network (DNN) model that includes an exemplar-replay-style continuous learning (CL) algorithm. The DNN model may be configured to receive information originating from a wearable medical sensor and receive replay data from the CL algorithm. The CL algorithm may be configured with a data preservation module and a synthetic data generation module. The data preservation module may be configured to preserve a subset of training data from one or more previous missions based on an average training loss of each data instance. The synthetic data generation module may be configured to model a probability distribution of real training data and then generate synthetic data sufficient for replays while maintaining data privacy.

In certain aspects, the processing unit(s) may be configured to perform a method. In certain aspects, the method may be a method for continually learning to detect new diseases with a single neural network model. The method may include generating data for a current mission by receiving data from a wearable medical sensor as disclosed herein, and preprocessing the received data. The method may include receiving data from previous missions from an exemplar-replay-style CL algorithm. The received data may be from a single mission, or from a plurality of missions. The method may include analyzing the data for the current mission to determine if more output neurons and/or detection heads are needed in a multi-headed deep neural network (DNN) model for the current mission.

If more output neurons and/or detection heads are needed, the method may include expanding the DNN model to include more output neurons and/or detection heads as needed. Techniques for adding output neurons or detection heads to a DNN model are well known in the art; any appropriate technique for doing so may be utilized.

The method may include training the DNN model jointly with substantially equal data from the current mission and previous missions. As used herein, the term “substantially equal data” generally refers to having data from multiple sources, where the amount of data from each source would reasonably be considered to be about evenly split between the data sources. For example, in some embodiments, the total number of values from each data source may be within ±20%, ±10%, ±5%, or ±2% of the average number of values from all data sources.

The method may include either preserving or generating data, be either (i) (a) obtaining the average training loss of each data instance in the current mission after training, (b) computing a threshold value for data preservation, and (c) preserving data instances whose average training loss values are above the threshold value for future replays; or (ii) (a) modeling a joint multivariate probability distributions of real training data through both parametric and non-parametric density estimation methods, and (b) sampling at least some synthetic data (e.g., as much synthetic data as required) from the joint multivariate probability distributions.

CL is usually defined as training a single ML model on a sequence of missions where non-stationary data distributions, different classification classes, or distinct tasks are presented sequentially. In addition, data from previous missions may no longer be available while training on current and future missions. A potentially infinite sequence of missions may be defined as M={M₁, M₂, . . . , Mₙ}, where the n-th mission is depicted by

$${M}_{n} = \{ ( {X}_{n}^{i} , {Y}_{n}^{i} ) \vert i = 1 , 2 , … , {C}_{n} \} . {X}_{n}^{i} ∈ 𝒳 and {Y}_{n}^{i} ∈ 𝒴$$

refer to the set of features and the corresponding class label for the i-th class in mission Mₙ. Cₙ refers to the total number of classes in mission Mₙ. The objective of CL is to train a single model f_{θ}:χ→ [symbol] , parameterized by θ, such that it can sequentially learn new missions in M and predict their labels Y=f_{θ}(X∈χ)∈ [symbol] without degrading the performance on the previous missions.

Depending on the mission transition scenario, CL can usually be categorized into three different settings: (i) domain-, (ii) class-, and (iii) task-incremental CL.

Domain-incremental CL represents the scenario where data from different distribution domains for the same task become available sequentially. In other words, mission Mₙ contains the same task and classification classes as missions [M₁:Mₙ₋₁], but

$${X}_{n}^{i} and [ {X}_{1}^{i} : {X}_{n - 1}^{i} ]$$

come from different probability distributions. For example, the model trained on the data collected from the elderly has to adapt to new data collected from young adults.

Class-incremental CL describes the setting where new classification classes of the current task emerge incrementally. To put it another way, mission Mₙ encloses the same task as missions [M₁:Mₙ₋₁], yet

$${Y}_{n}^{i}$$

includes classes unseen in

$$[ {Y}_{1}^{i} : {Y}_{n - 1}^{i} ] .$$

For instance, some patients are diagnosed with COVID-19 while not experiencing any symptoms and are later categorized as asymptomatic-positive.

Task-incremental CL refers to the case where new classification tasks appear in a sequence. That is, mission Mₙ contains new classification tasks that are unseen in missions [M₁:Mₙ₋₁]. For example, mission Mₙ may contain the COVID-19 detection task whereas missions [M₁:Mₙ₋₁] may comprise other disease detection tasks.

To evaluate the efficacy of DOCTOR, extensive experiments were conducted in all three settings with three different disease datasets obtained from commercially available WMSs. This is described in more detail later.

Several metrics are popularly used to evaluate CL algorithms, such as average accuracy, average forgetting, backward transfer, and forward transfer. To illustrate the overall multi-disease detection capabilities of DOCTOR, the average accuracy metric was selected to evaluate its performance on continually learning new disease detection missions while preserving knowledge of previously learned ones. In addition, it is essential to ensure the model does not raise many false alarms or misclassify disease-positive cases in disease detection missions. Therefore, the average macro F1-score across all learned missions was derived to evaluate the framework.

Average accuracy (Acc_{avg}) measures the average test accuracy of a CL model across all learned missions. We define aqn as the test accuracy of the n-th mission after continually learning a total of q missions, where n≤q. Then, Accavg can be defined as:

$${Acc}_{avg} = \frac{1}{q} {Σ}_{n = 1}^{q} {a}_{n}^{q} \qquad ( 1 )$$

F1-score calculates the harmonic mean of the precision and recall of a classification class. For each class, precision evaluates a model's performance with a focus on the number of false positive (FP) instances, whereas recall focuses on the number of false negative (FN) instances. The F1-score combines both metrics into a single number to evaluate how well the model suppresses both FP and FN in that class, as follows:

$$F 1 - score = 2 \times \frac{Precision \times Recall}{Precision + Recal} = \frac{TP}{TP + \frac{1}{2} ( FP + FN )} \qquad ( 2 )$$

To evaluate a model's overall performance in suppressing FP and FN, the macro F1-score calculates the arithmetic mean of the F1-scores of all classification classes. Therefore, to provide a general evaluation of DOCTOR, the average macro F1-score across all learned missions, defined as F1-score_{avg}, is reported. The macro F1-score of the n-th mission after continually learning q missions is defined as

$$F 1 {score}_{n}^{q} ,$$

where n≤q. Then, F1-score_{avg} can be defined as:

$$F 1 - {score}_{avg} = \frac{1}{q} {\sum }_{n = 1}^{q} F 1 {score}_{n}^{q} \qquad ( 3 )$$

In addition, to evaluate the proposed replay-style CL algorithm for mitigating catastrophic forgetting, the BWT value was also reported in the experimental results. When learning a new mission, BWT measures how much the CL algorithm impacts the performance of the framework on previous missions. BWT can be defined as:

$$BWT = \frac{1}{q - 1} {\sum }_{n = 1}^{q - 1} ( {a}_{n}^{q} - {a}_{n}^{n} ) \qquad ( 4 )$$

where

$${a}_{n}^{q}$$

represents the test accuracy of the framework for the n-th mission after continually learning a total of q missions, and

$${a}_{n}^{n}$$

denotes the test accuracy of the framework for the n-th mission after continually learning a total of n missions.

The disclosed framework operates directly on tabular data collected from commercially available WMSs without the need for manual feature extraction. It is capable of learning multiple disease detection tasks sequentially with a single DNN model through a proposed replay-style CL algorithm. The CL algorithm relies on a data preservation method and a synthetic data generation module. When the DNN model continually learns new missions, the CL algorithm retrieves data from previous missions and replays them with the new data to fine-tune the model. Finally, the DNN model features a multi-head architecture that generates individual classification probability distribution for each output detection head. Therefore, DOCTOR can simultaneously detect multiple diseases for patients based on their WMS data.

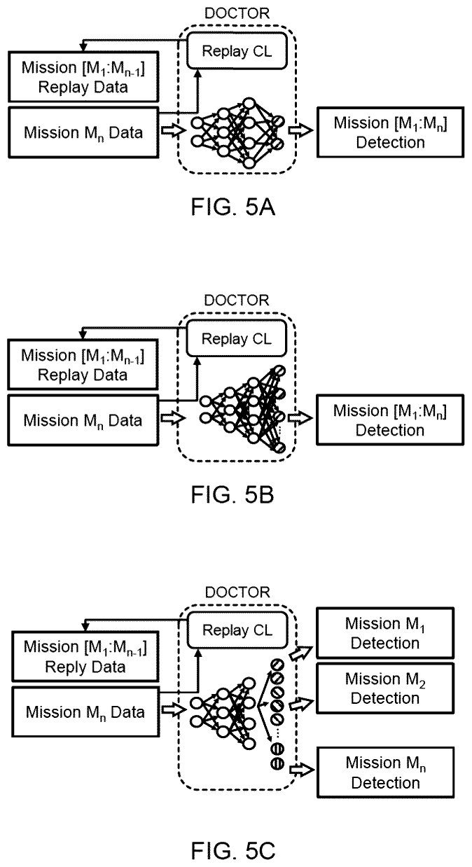

FIG. 4 illustrates the top-level flowchart of DOCTOR operating in a CL mission. First, the framework collects physiological signals from patients through commercially available WMSs. Then, basic data preprocessing is applied to the raw data, including data stream synchronization and windowing, to prepare the datasets. Next, the CL algorithm gathers data from previous missions for replay during training. In the model expansion step, the framework analyzes if more output neurons or detection heads are required for the current mission and expands the DNN model accordingly (discussed below). Due to differences in training dataset sizes across missions, the model might be biased toward missions that have more available training data. To avoid the biased prediction problem, the model is trained jointly with data from the current mission and previous missions in a balanced fashion. That is, an even amount of data is gathered from each mission to form a mini-batch for training. In parallel, the user or practitioner can specify which method to use in the replay-style CL algorithm. If preserving real data is allowed for the current mission and memory storage is sufficient, the efficient data preservation method can be applied. It obtains the average training loss of each data instance in the current mission after training and computes the threshold value for data preservation. Then, it preserves data instances whose average training loss values are above the threshold value for future replays (discussed below). If patient privacy is critical to the current mission, the synthetic data generation module can be used to preserve data privacy. It models the joint multivariate probability distributions of the real training data through both parametric and non-parametric density estimation methods. Then, the module samples as much synthetic data as required from the learned probability distributions (discussed below).

Next, the multi-disease detection workflows and the DNN model expansion procedures of DOCTOR are discussed under three different CL scenarios. Details of how the framework continually adapts to data distribution domain shifts and new classification classes, and learns to perform new disease detection tasks, are given. Then, its application to multi-disease detection at test time is explained.

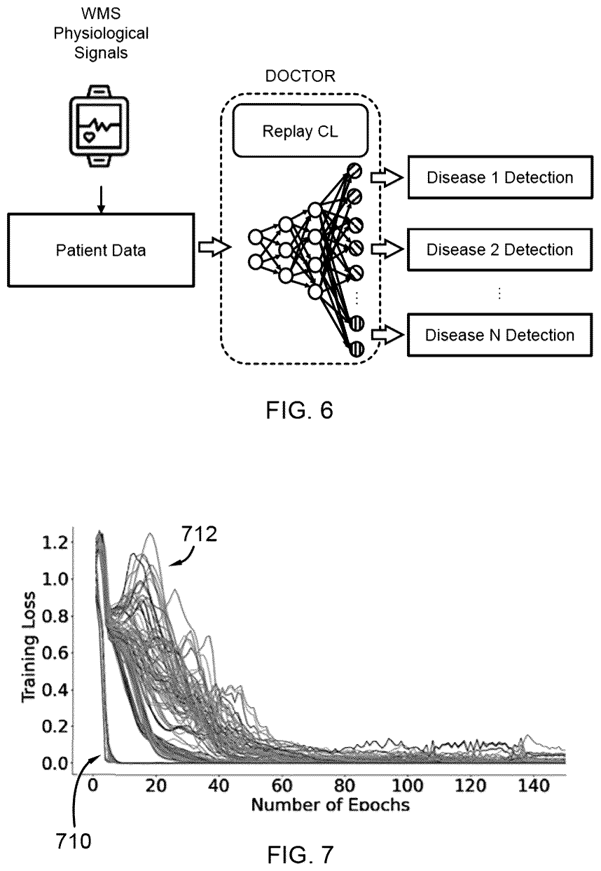

Domain-incremental Scenario: FIG. 5A shows the workflow of DOCTOR in the domain-incremental CL scenario. As described previously, the new mission Mₙ contains the same task and classification classes as missions [M₁:Mₙ₋₁] in this setting and DOCTOR sequentially adapts to data from different domains. For example, data from different cities, countries, or even age groups become available incrementally for the same disease. Thus, the framework does not need to add new output neurons or create new output detection heads to accommodate new missions in the model expansion step. The DNN model just needs to be fine-tuned jointly with the data from the new domain and previous domains to prevent catastrophic forgetting.

Class-incremental Scenario: FIG. 5B illustrates the workflow of DOCTOR in the class-incremental CL scenario. In this setting, the new mission Mₙ includes unseen classification classes in missions [M₁:Mₙ₋₁] for the same task, as mentioned previously. For instance, various types of patients may be discovered incrementally for the same disease. Hence, the framework needs to add new output neurons to the DNN model to accommodate new classification classes. In addition, the DNN model inherits the trained weights from the last stage after model expansion. Then, the model gets trained jointly with data from the new mission and previous missions to learn the new classification classes and recall the previous ones.

Task-incremental Scenario: FIG. 3C demonstrates the workflow of DOCTOR in the task-incremental CL scenario. As explained previously, the new mission Mₙ includes a different task with completely distinct classification classes from missions [M₁:Mₙ₋₁] in this scenario. Hence, DOCTOR incrementally learns to detect various disease detection tasks, such as COVID-19, diabetes, and mental health disorders. Therefore, a multi-head architecture is adopted for the DNN model to accommodate new disease detection tasks in task-incremental scenarios. As shown in FIG. 5C, when a new disease detection task arrives, the framework generates a new detection head in the output layer of the DNN model in the model expansion step. The new detection head is added in parallel to the other ones. Therefore, it can learn to detect the new disease and perform classification individually without interfering with the other heads. As before, the DNN model inherits the trained weights for the learned tasks from the last stage after expansion. Then, the model is trained with the data from the new task and fine-tuned with the data from previous tasks through an exemplar replay at their corresponding detection heads. A softmax layer is adopted for each output detection head. Hence, each head can generate an individual probability distribution for classification and output the predicted detection result for each disease separately. The cross-entropy loss at each head is then obtained for its corresponding training data instances and their true labels. Lastly, the losses from all detection heads are accumulated to perform backpropagation.

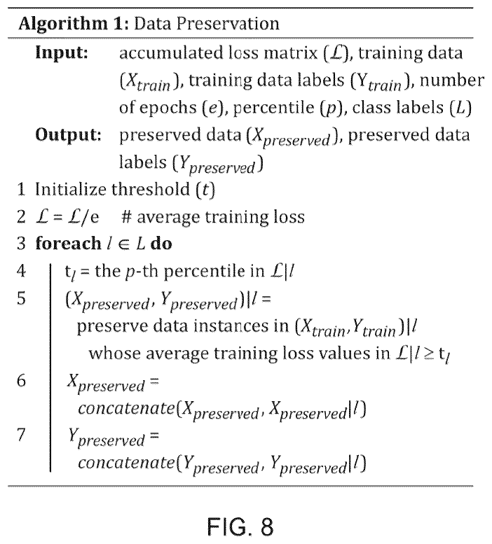

Multi-disease Detection Application: The multi-head architecture allows DOCTOR to generate individual classification probability distribution for each detection head. Therefore, the disclosed framework can perform detection for different diseases in parallel. FIG. 6 demonstrates how DOCTOR can be used for multi-disease detection. At test time, physiological signals are collected with commercially available WMSs and sent to the framework. The framework then preprocesses the raw data and performs disease detection with the multi-head DNN model. Finally, DOCTOR outputs the detection result for each disease from each detection head. Therefore, patients can be informed about which diseases are detected from their physiological signals with a single run of examination. For example, a patient may learn that he or she is asymptomatic-positive for COVID-19, Type-2 diabetic, and depressed through a single test.

The CL algorithm proposed for the DOCTOR framework targets the tabular data domain and counteracts catastrophic forgetting in an exemplar-replay manner. It consists of a data preservation method and a synthetic data generation module. Based on the use scenario and user settings, the CL algorithm can preserve the most informative subsets of real training data from previous missions or generate synthetic data from the learned multivariate probability distributions of past missions for future replays.

Data Preservation: In scenarios when computational resources are limited and preserving actual data is not prohibited, DOCTOR adopts the data preservation method to preserve subsets of real training data and their labels from the previous missions for future replays. The method samples the most informative subsets from the real training data in a stratified fashion based on the average training loss value of each training data instance. Then, the preserved data are replayed together with data from the new mission in future CL scenarios to recall the previous missions.

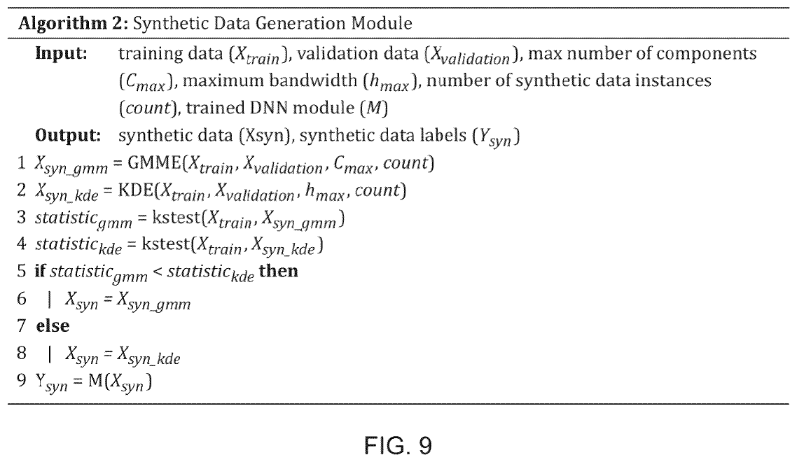

Inspired by a framework called CTRL, the average training loss of each data instance is considered to evaluate how informative the instance is. FIG. 7 shows the graph of the training loss of each training data instance in the CovidDeep dataset across 150 epochs. The training losses of some data instances (e.g., data instances (710)) decrease fast and stay near zero after around 40 epochs. These data instances have low average training loss values and are easy for the DNN model to learn. Therefore, they do not contain sufficient information to serve as good exemplars for the model to recall the mission. On the other hand, the data instances with higher average training loss values (e.g., data instances (712)) are more informative about the mission. Hence, they are good candidates to be preserved for the model to recall the mission in future replays.

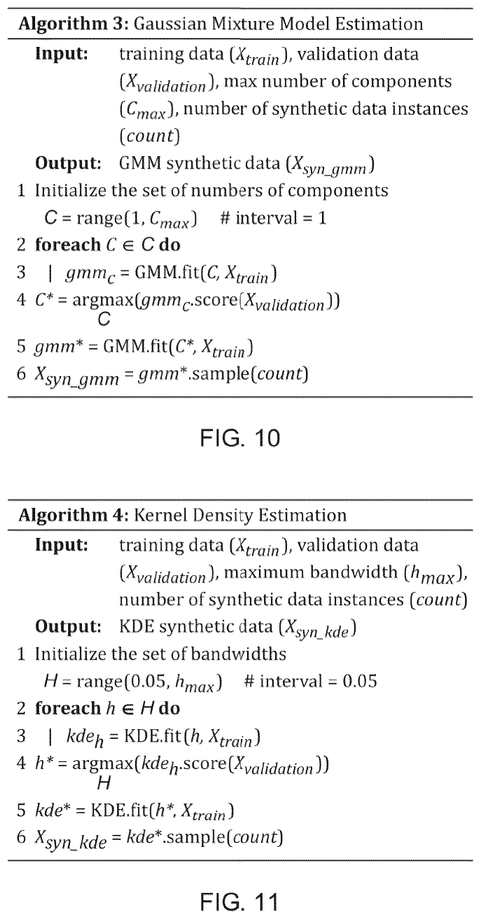

Algorithm 1 (see FIG. 8) provides the pseudocode of the data preservation method in our replay-style CL algorithm. During the model training process, DOCTOR accumulates the training loss of each training data instance of the current mission in a loss matrix [symbol] after each epoch. After training finishes, the data preservation method first calculates the average training loss of each data instance by dividing [symbol] by the number of epochs e. Then, for each class label l, the algorithm sets a threshold value tₗ as the p-th percentile of the average training loss values in [symbol] |l. Next, the algorithm preserves the data instances X_{preserved}|l and their corresponding labels Y_{preserved}|l whose average training loss values in [symbol] |1 are greater than or equal to their corresponding threshold values tₗ. Finally, the preserved data X_{preserved} and their corresponding labels Y_{preserved} are obtained by concatenating all X_{preserved}|l and Y_{preserved}|l respectively. Therefore, the data preservation method preserves the most informative subset of the real training data for the current mission in a stratified manner without excessive computation. The preserved data can then be used to replay the mission in future CL scenarios.
2) Synthetic Data Generation Module Overview: In real-world scenarios for disease detection applications, data privacy should be taken into account, and preserving actual patient data may often be prohibited. In such cases, DOCTOR adopts a synthetic data generation module to generate synthetic data for future replays while preserving data privacy. The module first models the joint multivariate probability distribution of the real training data of the current mission. Next, it generates synthetic data by sampling from the learned distribution. Then, DOCTOR labels the synthetic data with the trained DNN model and replays them in future CL scenarios to recall the previous missions.

Algorithm 2 (see FIG. 9) depicts the top-level pseudocode of the synthetic data generation module. The module uses probability density estimation methods to model the probability density function (PDF) of the joint multivariate probability distribution of the given real training data Xₜᵣₐᵢₙ. Predominantly, probability density estimation methods can be categorized into two groups: parametric and non-parametric. Whereas a PDF is assumed to be a member of a parametric family in the former, there are no assumptions made for it in the latter. The parametric Gaussian mixture model estimation (GMME) method GMME ( ) and the non-parametric kernel density estimation (KDE) method KDE ( ) are implemented in the synthetic data generation module. First, the module generates synthetic data X_{syn_gmm} and X_{syn_kde} from the GMME ( ) and KDE ( ) functions, respectively. Next, to decide which synthetic data to use for future replays, the two-sample Kolmogorov-Smirnov (KS) test kstest ( ) is applied to X_{syn_gmm} and X_{syn_kde}. The KS test statistic is used to evaluate the closeness of the underlying distributions of the two given samples. A lower KS statistic number shows a higher closeness of the distributions. Therefore, the synthetic data with a lower KS test statistic was chosen. Finally, DOCTOR generates labels Y_{syn} for the chosen X_{syn} with the trained DNN model M. The synthetic data X_{syn} and corresponding labels Y_{syn} are then used to replay the mission in future CL scenarios.
3) The Gaussian Mixture Model Estimation Method: The GMME method utilizes a multi-dimensional Gaussian mixture model (GMM) to model the probability distribution of the given real training data. Its PDF is modeled as a mixture of C Gaussian models in the form of:

$${p}_{X} ( x \vert Θ ) = {Σ}_{c = 1}^{C} {p}_{X \vert Z} ( x \vert z = c , Θ ) {p}_{Z} ( z = c \vert Θ )$$

where Θ represents the parameters of the Gaussian model, X depicts the observed variables, and Z symbolizes the hidden state variables that indicate the Gaussian model assignment. The prior probability of each model component c can be written as:

$${p}_{Z} ( z = c \vert Θ ) = {θ}_{c} .$$

Each Gaussian model component c is a d-dimensional multi-variate Gaussian distribution with mean vector μ_{c} and covariance matrix Σ_{c} in the form of:

$${P}_{X \vert Z} ( x \vert z = c , Θ ) = {( \frac{1}{2 π} )}^{\frac{d}{2}} {\vert {Σ}_{c} \vert}^{- \frac{1}{2}} exp ( - \frac{1}{2} {( x - {μ}_{c} )}^{T} {Σ}_{c}^{- 1} ( x - {μ}_{c} ) ) ,$$

where |Σ_{c}| is the determinant of the covariance matrix. Therefore, the PDF modeled by GMM can be simplified as follows:

$${P}_{X \vert C} ( x ) = {Σ}_{c = 1}^{C} {π}_{C} 𝒩 ( x \vert {μ}_{c} , {Σ}_{c} ) ,$$

where π_{c} represents the weight of the Gaussian model component c. To alleviate its learning complexity issue, the iterative expectation-maximization (EM) algorithm is used to determine the GMM parameters.

Algorithm 3 (see FIG. 10) shows the pseudocode of the GMME method GMME ( ). The algorithm starts by initializing a set [symbol] of candidate numbers for the total number of Gaussian model components ranging from 1 to a user-specified maximum number Cₘₐₓ. Then, for each candidate number C in [symbol] , the algorithm fits a GMM gmm_{C} to the given real training data Xₜᵣₐᵢₙ with C Gaussian model components. Next, to prevent the GMM from overfitting on Xₜᵣₐᵢₙ, the total number of Gaussian models C needs to be chosen appropriately. Hence, the algorithm computes the per-sample average log-likelihood gmm_{C}.score ( ) of the given real validation data X_{validation} under each gmm_{C} to evaluate the quality of the model. The number C that maximizes this criterion is chosen as the optimal total number of C* Gaussian models required to model the probability distribution of Xₜᵣₐᵢₙ. Finally, the algorithm finalizes the GMM gmm* with parameter C* and Xₜᵣₐᵢₙ, and samples a user-defined number count of synthetic data X_{syn_gmm} from gmm*.
4) The Kernel Density Estimation Method: The KDE method approximates the probability distribution of the given real training data as a sum of many designated kernel functions. Each kernel function K should satisfy the following property:

$${\int }_{- ∞}^{∞} K ( x ) d x = 1 , K ( x ) > 0 ∀ x$$

In addition, another important design parameter for the KDE method is the kernel bandwidth h that scales K and controls the smoothness of the estimated function. In general, the approximated PDF \hat{f} with the KDE method can be formulated as follows:

$$\hat{f} ( x ) = \frac{1}{N h} {Σ}_{i = 1}^{N} K ( \frac{x - {x}_{i}}{h} ) ,$$

where N is the total number of samples and xᵢ is the i-th sample. The kernel bandwidth h has a strong impact on the bias-variance trade-off of the KDE. Essentially, high-variance models estimate the training data well but suffer from overfitting on the noisy training data. On the other hand, high-bias models are simpler but suffer from underfitting on the training data. Whereas h has to be close to 0 to achieve a small bias, h needs to be close to ∞ to achieve a small variance. The PDF of the normal distribution was chosen as the kernel function. Therefore, the PDF of the given real training data can be formulated as follows:

$$p ( x \vert Θ ) = {Σ}_{i = 1}^{N} {p}_{Z} ( {z}_{i} = c \vert Θ ) {p}_{X \vert Z} ( x \vert z = c , Θ ) ,$$

where Θ represents the parameters of the normal distribution, X depicts the observed variables, and Z symbolizes the hidden state variables that indicate the normal distribution assignment.

Algorithm 4 (see FIG. 11) shows the pseudocode of the KDE method KDE ( ). First, the algorithm initializes a set [symbol] of candidate values for the kernel bandwidth ranging from 0.05 to a user-specified maximum bandwidth hₘₐₓ. Then, for each candidate bandwidth h in [symbol] , the algorithm fits a KDE model kdeₕ on the given real training data Xₜᵣₐᵢₙ with bandwidth h. Next, as discussed earlier, the algorithm finds the appropriate kernel bandwidth value for the final KDE model. The algorithm computes the total log-likelihood kdeₕ.score ( ) of the given real validation data X_{validation} under each kdeₕ to evaluate the quality of the model. The value h that maximizes this criterion is designated as the final kernel bandwidth h* required to model the probability distribution of Xₜᵣₐᵢₙ. Finally, the algorithm fits the final KDE model kde* with parameter h* and Xₜᵣₐᵢₙ, and samples a user-defined number count of synthetic data X_{syn_kde} from kde*.

To evaluate the efficacy of DOCTOR in continually learning various disease detection missions, we conduct experiments with three different disease datasets: CovidDeep, DiabDeep, and MHDeep.

The CovidDeep dataset includes physiological signals and responses to a simple questionnaire acquired from 38 healthy individuals, 30 asymptomatic patients, and 32 symptomatic patients at San Matteo Hospital in Pavia, Italy. The data were collected with commercially available WMSs and devices, including an Empatica E4 smartwatch, a pulse oximeter, and a blood pressure monitor. The DiabDeep dataset contains physiological signals and demographic information obtained from 25 non-diabetic individuals, 14 Type-1 diabetic patients, and 13 Type-2 diabetic patients. The data were collected with an Empatica E4 smartwatch and a Samsung Galaxy S4 smartphone. The MHDeep dataset contains physiological data obtained from 23 healthy participants, 23 participants with bipolar disorder, 10 participants with major depressive disorder, and 16 participants with schizoaffective disorder at the Hackensack Meridian Health Carrier Clinic, Belle Mead, New Jersey. The data were collected with an Empatica E4 smartwatch and a Samsung Galaxy S4 smartphone.

All three datasets were preprocessed before using them in the experiments. To avoid time correlation between adjacent data windows, the data streams were first synchronized and windowed by dividing data into 15-second windows with 15-second shifts in between. Each 15-second window of data constitutes one data instance. Next, we flatten and concatenate the data within the same time window from the WMSs and smartphones. Then, the sequential time series data was concatenated with the responses to the questionnaire for the CovidDeep data. This results in a total of 14,047 data instances with 155 features each for the CovidDeep dataset, a total of 20,957 data instances with 4,485 features each for the DiabDeep dataset, and a total of 27,082 data instances with 4,485 features each for the MHDeep dataset. Following this, min-max normalization was performed on the feature data in all datasets to scale them into the range between 0 and 1. This prevents features with a wider range of values from overshadowing those with a narrower range.

To align the input feature dimensions of all three datasets for CL experiments, principal component analysis was applied to the DiabDeep and MHDeep datasets to reduce their dimensionality from 4485 to 155. First, the feature data in the datasets and the covariance matrix of the features were standardized and computed. Then, eigendecomposition was performed to find the eigenvectors and eigenvalues of the covariance matrix to identify the principal components. Next, the eigenvectors were ordered in descending order based on the magnitude of their corresponding eigenvalues. Finally, a projection matrix was constructed to select the top 155 principal components. Then, the data was recast along the principal component axes to retrieve the 155-dimensional feature data for these datasets. Subsequently, the datasets were partitioned into training, validation, and test sets based on different CL scenarios with no time overlap.

Last but not least, the Synthetic Minority Oversampling Technique (SMOTE) was applied to the partitioned training datasets to counteract the data imbalance issue within each dataset. This was started by selecting a random data instance A in a minority class and finding its five nearest neighbors in that class. Then, one nearest neighbor B was randomly selected from the five and a line segment was drawn between A and B in the feature space. Finally, a synthetic data instance is generated at a randomly selected point on the line segment between A and B. This process was repeated until a balanced number of data instances in all classes in each partitioned training set was obtain.

DOCTOR learns various disease detection tasks incrementally in the sequential time series tabular data domain with a single DNN model and the proposed replay-style CL algorithm. Many DNN models can be employed in the framework and perform classification tasks well on sequential time series data, such as recurrent neural networks (RNNs) and long short-term memory (LSTM) networks. However, these networks are difficult to train and require more training data and computational resources. Thus, a much preferred embodiment was implemented using a multi-layer perceptron (MLP) model in our framework for the experiments.

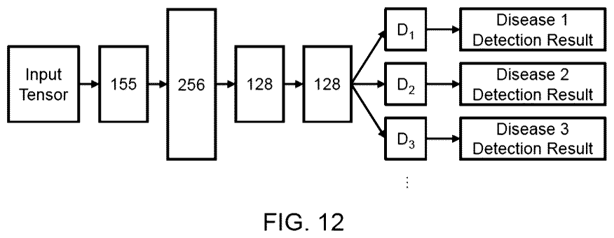

FIG. 12 shows the architecture of the MLP model. It has an input layer with 155 neurons to align the number of input features. It has three hidden layers with widths set to 256, 128, and 128 neurons. In some embodiments, the exact number of neurons in each layer may vary. In some embodiments, the ratio of widths in the three layers is 2:1:1 neurons.

Finally, it incorporates a multi-head architecture in the output layer to accommodate each disease detection task that is learned. An output head has a varying number of neurons corresponding to the number of classification classes in that task. It uses the rectified linear unit (ReLU) as the nonlinear activation function in the hidden layers and the softmax function for each head in the output layer to generate detection results.

The stochastic gradient descent (SGD) optimizer is used with a momentum of 0.9 in these experiments and the learning rate is initialized to 0.005. The batch size is set to 128 for training, where data are drawn evenly from the current mission and each previous mission. The MLP model is trained for 300 epochs. In some embodiments, the MLP model may be trained for 150-1000 epochs. For the data preservation method, the threshold value is set as the 70th percentile of the average training loss values in each classification class. For the synthetic data generation module, the maximum number of components is set to 50 for the GMME method and the maximum bandwidth to 0.5 for the KDE method. Finally, the same number of synthetic data instances are sampled from the module as the number of real training data instances.

DOCTOR may be implemented with, e.g., PyTorch and the experiments may be performed on any appropriate processing unit (in these examples, an NVIDIA A100 GPU was used). CUDA and cuDNN libraries were employed to accelerate the experiments.

Each CL experiment is repeated three times and the average values for all evaluation metrics are reported. Also reported are the results obtained using a naive fine-tuning framework to depict a performance lower bound and a joint-training framework to depict a performance upper bound. The naive fine-tuning framework fine-tunes the MLP model with only the new data when a new mission arrives. The joint-training framework assumes that the framework always has full access to all the data from previous missions and trains the MLP model jointly with all the data from the new and previous missions. Experimental results for the MLP model after being trained in the first mission are also reported as the performance baseline.

### A. Domain-Incremental Continual Learning Scenario

In the domain-incremental CL experiments, two missions were assume where data from two different distributions for the same disease detection task become available sequentially. For all three datasets, the patients were first randomly split into two missions (groups) in a stratified fashion, where Mission 1 (M₁) has 80% of the patients from each class and Mission 2 (M₂) has the remaining 20%. Then, within each mission, the first 70%, the next 10%, and the last 20% of each patient's sequential time series data were taken to construct the training, validation, and test sets with no time overlap. Subsequently, SMOTE was applied to the training sets in both missions to counteract the data imbalance issue.

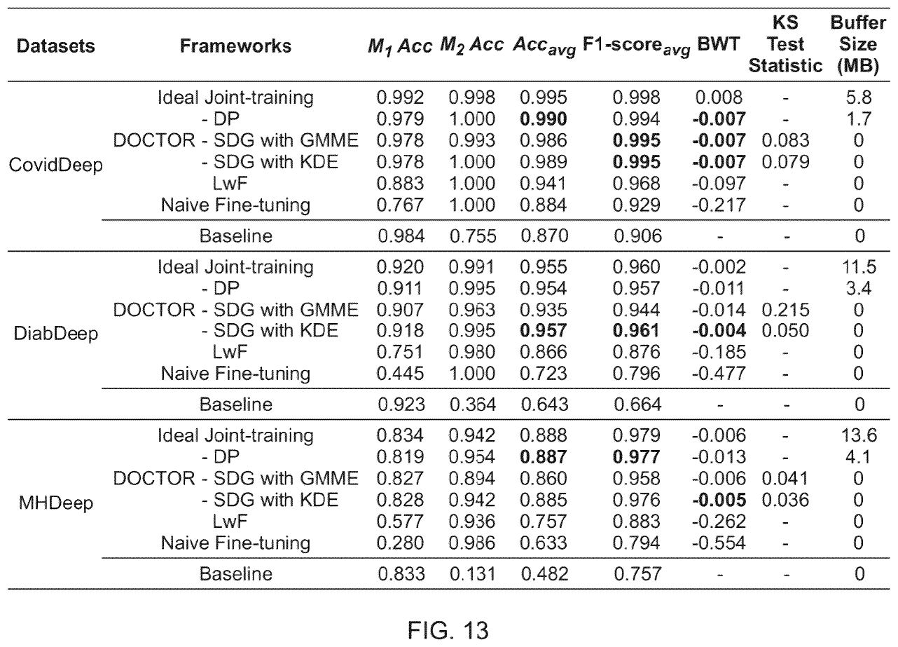

In the domain-incremental scenario, new missions just contain data from different probability distributions but with the same classification classes as the given task. Hence, the MLP models do not need to expand their output layers to accommodate new missions. Table 3 (see FIG. 13) shows the experimental results for all frameworks in the domain-incremental CL scenario. As one can see from the table, for all three datasets, the MLP model suffers from significant performance deterioration for M₁ when the naive fine-tuning framework is used, due to catastrophic forgetting. This results in poor Acc_{avg}, F1-score_{avg}, and BWT compared to the other frameworks. DOCTOR far outperforms the naive fine-tuning and LwF frameworks, and achieves very competitive Acc_{avg}, F1-score_{avg}, and BWT relative to the ideal joint-training framework for all three datasets. Moreover, DOCTOR even outperforms the ideal scenario in some cases due to the generalization gained through learning from synthetic data. Next, the KS test statistics was examined when DOCTOR employs generative replay with the SDG module. When GMME and KDE both yield similar KS test statistics for the CovidDeep and MHDeep datasets, DOCTOR achieves a very similar test accuracy for M₁ with the synthetic data generated using each method. However, for the DiabDeep dataset, the KDE method results in a lower KS test statistic and hence a higher test accuracy for M₁. This validates the design decision in choosing the estimation method that yields a lower KS test statistic for generative replay.

In summary, DOCTOR achieves a 0.990 Acc_{avg}, a 0.995 F1-score_{avg}, and a −0.007 BWT on the CovidDeep dataset, a 0.957 Acc_{avg}, a 0.961 F1-score_{avg}, and a −0.004 BWT on the DiabDeep dataset, and a 0.887 Acc_{avg}, a 0.977 F1-score_{avg}, and a −0.005 BWT on the MHDeep dataset. This demonstrates that DOCTOR can incrementally adapt to data from new distributions while maintaining the knowledge learned from previous ones. The best DOCTOR results are shown in bold.

### B. Class-Incremental Continual Learning Scenario

In the class-incremental CL experiments, two missions were assume where M₂ contains new classification classes not seen in M₁ for the same disease detection task. For CovidDeep, only the healthy individuals and symptomatic patients in M₁ were included, whereas M₂ only contains the asymptomatic patients. For DiabDeep, M₁ includes the healthy individuals and Type-1 diabetic patients, whereas M₂ comprises solely the Type-2 diabetic patients. For MHDeep, the healthy participants and participants with major depressive disorder in M₁ were included, whereas M₂ consists of participants with bipolar depressive disorder and schizoaffective disorder. As before, within each mission, the training, validation, and test sets were prepared by taking the first 70%, the next 10%, and the last 20% of the sequential time series data instances from each patient with no time overlap. Similarly, SMOTE was applied to the training sets in both missions to address the data imbalance issue.

In the class-incremental CL scenario, the MLP models in all frameworks except for the baseline framework need to add more output neurons to accommodate the unseen classification classes. Therefore, in those frameworks, they first inherit the weights of their MLP models from the baseline framework and then add the same number of new output neurons as the number of unseen classes in M₂. Then, the MLP models are trained with various CL methods corresponding to their frameworks.

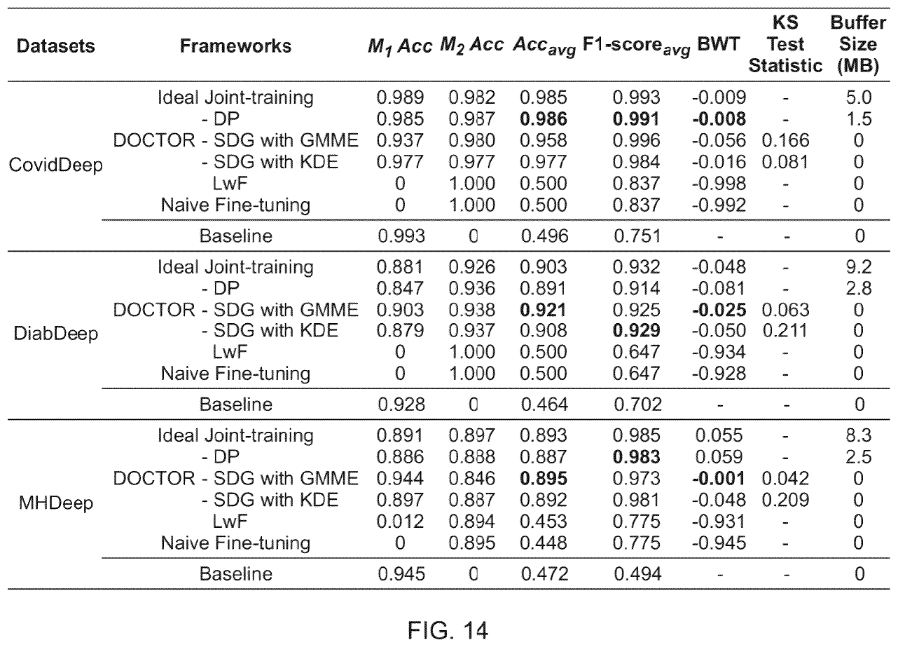

Table 4 (see FIG. 14) presents the experimental results for all frameworks in the class-incremental CL scenario. As can again be seen from the table, due to catastrophic forgetting, the MLP model suffers from significant performance degradation on M₁ when the naive fine-tuning framework is used in all three datasets. However, DOCTOR significantly outperforms the naive fine-tuning and LwF frameworks. Moreover, due to fine-tuning with the most informative preserved data and the generalization gained through learning from synthetic data, it even achieves higher Acc_{avg} and BWT and a competitive F1-score_{avg} relative to the ideal joint-training framework for all three datasets. On the other hand, as shown in the table, the estimation method that yields a lower KS test statistic results in a better test accuracy for M₁ for all datasets. This again validates the design decision made regarding the KS test statistic.

To summarize, DOCTOR achieves a 0.986 Acc_{avg}, a 0.991 F1-score_{avg}, and a −0.008 BWT on the CovidDeep dataset, a 0.921 Acc_{avg}, a 0.929 F1-score_{avg}, and a −0.025 BWT on the DiabDeep dataset, and a 0.895 Acc_{avg}, a 0.983 F1-score_{avg}, and a −0.001 BWT on the MHDeep dataset. This demonstrates the efficacy of DOCTOR in learning new classification classes while preserving prior knowledge. Again, the best DOCTOR results are shown in bold.

### C. Task-Incremental Continual Learning

In the task-incremental CL experiments, three missions were assigned where DOCTOR learns three different disease detection tasks incrementally with the three disease datasets. First, DOCTOR was allowed learn to detect the COVID-19 virus among healthy, symptomatic, and asymptomatic patients using the CovidDeep dataset (M₁). Next, DOCTOR was allowed to learn to differentiate between healthy, Type-I diabetic, and Type-II diabetic patients using the DiabDeep dataset (M₂). Finally, DOCTOR was allowed to learn to recognize participants that are healthy or suffer from bipolar, major depressive, or schizoaffective disorder using the MHDeep dataset (M₃). For each mission, the training, validation, and test sets were constructed by taking the first 70%, the next 10%, and the last 20% of the sequential time series data instances from each patient in the disease dataset with no time overlap. Finally, SMOTE was applied to the training sets in all three missions to handle the data imbalance issue in the datasets.

In the baseline framework, the MLP model only contains a single detection head in the output layer and is only trained with CovidDeep data. The CovidDeep detection head has the same number of output neurons as the number of classes in the CovidDeep detection task. When M₂ arrives, all the other frameworks first inherit the weights of their MLP models from the baseline framework. Then, they generate a new detection head parallel to the CovidDeep detection head in the output layer. The new head has the same number of output neurons as the number of classes in the DiabDeep detection task. Then, the MLP models are trained accordingly with respect to their CL frameworks.

When M₃ becomes available, all frameworks except the baseline inherit the weights of their MLP models from their previous states, respectively. Then, they generate a new detection head in parallel with the CovidDeep and DiabDeep detection heads in the output layer. The newly generated head contains the same number of neurons as the number of classes in the MHDeep detection task. Then, the MLP models learn the new detection task according to their associated CL frameworks.

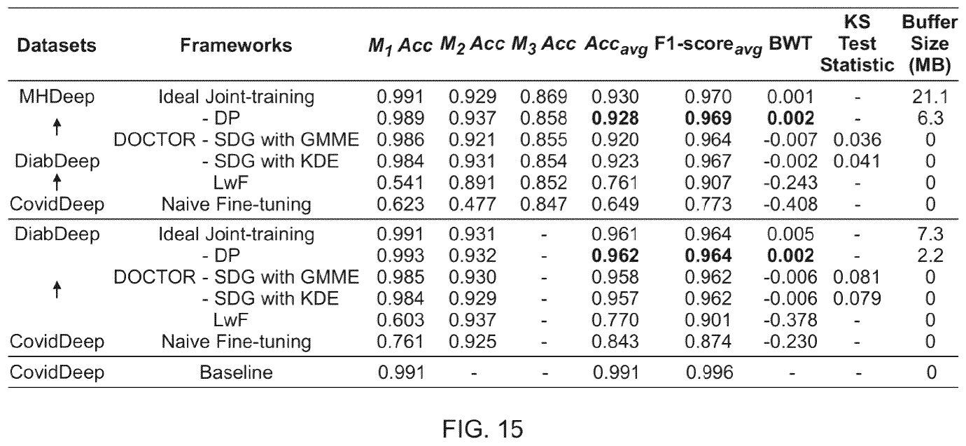

Table 5 (see FIG. 15) shows the experimental results for all frameworks in the task-incremental CL scenario. As shown in the table, due to catastrophic forgetting, the naive fine-tuning framework consistently suffers from performance deterioration in the previously learned detection tasks after learning a new task. The disclosed DOCTOR framework consistently outperforms both the naive fine-tuning and LwF frameworks. It achieves very competitive performance relative to the ideal joint-training framework even after incrementally learning multiple disease detection tasks. Moreover, DOCTOR is able to maintain high test accuracy for all the learned detection tasks in the task-incremental CL scenario. Both GMME and KDE methods yield similar KS test statistics in this scenario. Thus, DOCTOR achieves very similar test accuracy for M₁ and M₂ with the synthetic data generated from both estimation methods.

DOCTOR achieves a 0.962 Acc_{avg}, a 0.964 F1-score_{avg}, and a 0.002 BWT after continually learning two disease detection tasks, and a 0.928 Acc_{avg}, a 0.969 F1-score_{avg}, and a 0.002 BWT after consecutively learning to detect three diseases. This demonstrates DOCTOR's efficacy in the task-incremental scenario. The best DOCTOR results are again shown in bold.

The initial model size of DOCTOR after learning the CovidDeep dataset is 342 KB. The size increases to 344 KB after learning to detect the DiabDeep dataset due to the addition of three output neurons in the new detection head. Finally, the size increases to 346 KB after learning the MHDeep dataset due to the addition of another detection head with four output neurons. This allows DOCTOR to fit in various edge devices and makes it a promising application for efficient and out-of-clinic disease detection.

### D. Multi-Disease Detection

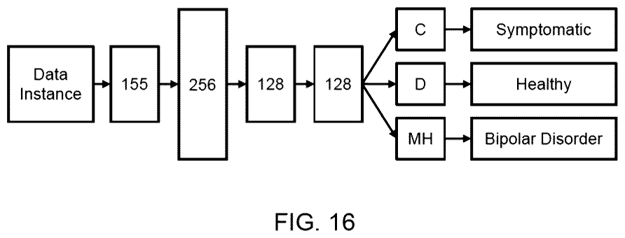

Next, the potential of simultaneous multi-disease detection with the DOCTOR framework is demonstrated. FIG. 16 illustrates an instance of simultaneous multi-disease detection at test time. For this demonstration, the MLP model trained with KDE-based synthetic data generation in the task-incremental CL experiment was used. First, one data instance of a symptomatic patient in the CovidDeep dataset was input to the multi-headed model. It informs that the patient is symptomatic-positive for COVID-19, healthy for diabetes, and positive for bipolar disorder. This can alert patients to seek further medical advice.

### E. Ablation Study

Finally, an ablation study of the data preservation CL algorithm was conducted. The domain-incremental CL experiments was repeated with the same settings as those in Section disclosed above for all three datasets. However, the percentile hyperparameter was modified to preserve different subsets of the real training data. The experiment was repeated three times while preserving the data instances whose average training loss values are above the 50th between the 75th and 25th and below the 50th percentile to preserve the top 50%, middle 50%, and bottom 50% subsets of the real training data, respectively.

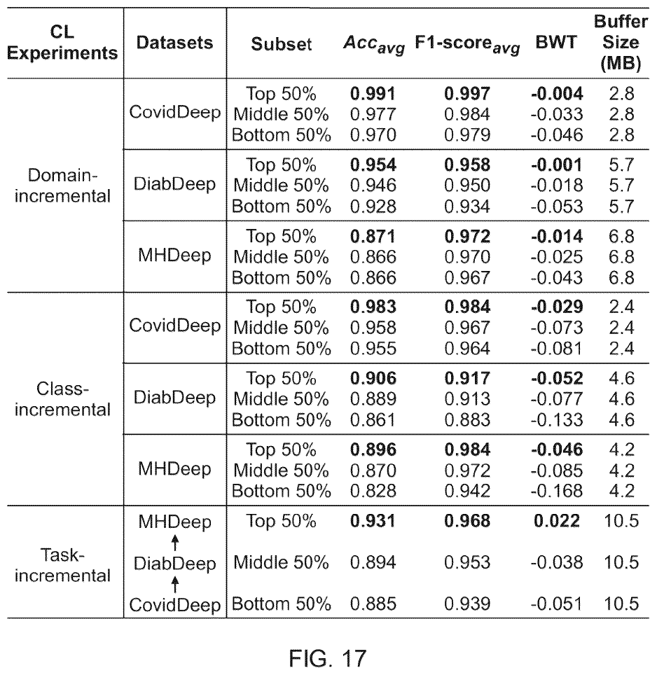

FIG. 17 shows the experimental results of the ablation study. Preserving the subset of the real training data whose average training loss values are in the top 50% results in a better performance for both CovidDeep and DiabDeep datasets. Even though preserving the middle 50% of the real training data in the MHDeep dataset gives higher Acc_{avg} and F1-score_{avg}, preserving the top 50% still achieves a very competitive performance. Therefore, it was chosen to preserve the subset of real training data whose average training loss values are above the 70th percentile for the data preservation CL algorithm in the disclosed framework.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined of the claims.

What is claimed is:

1. A non-transitory computer-readable storage device containing instructions that, when executed by one or more processing units, causes the one or more processing units to be configured to, either individually or collectively, deploy a multi-headed deep neural network (DNN) model that includes an exemplar-replay-style continuous learning (CL) algorithm;
wherein the DNN model is configured to receive information originating from a wearable medical sensor and receive replay data from the CL algorithm; and
wherein the CL algorithm is configured with a data preservation module and a synthetic data generation module;
wherein the data preservation module is configured to preserve a subset of training data from one or more previous missions based on an average training loss of each data instance; and
wherein the synthetic data generation module is configured to model a probability distribution of real training data and then generate synthetic data sufficient for replays while maintaining data privacy.

2. The non-transitory computer-readable storage device of claim 1, wherein a model size of the multi-headed DNN model is less than 1 MB.

3. The non-transitory computer-readable storage device of claim 2, wherein the model size is less than 500 KB.

4. The non-transitory computer-readable storage device of claim 1, wherein a model size of the multi-headed DNN model increases by less than 2% for each additional detection head that is added to the multi-headed DNN model.

5. The non-transitory computer-readable storage device of claim 1, wherein the synthetic data generation module is configured to model a joint multivariate probability distributions of real training data through both parametric and non-parametric density estimation methods, and generate synthetic data based on the learned probability distribution.

6. A system for continually learning to detect new diseases with a single neural network model, comprising:
a wearable medical sensor;
a non-transitory computer-readable storage device according to claim 1; and
one or more processing units operably coupled to the wearable medical sensor and to the non-transitory computer-readable storage device.

7. A method for continually learning to detect new diseases with a single neural network model, comprising:
generating data for a current mission by receiving data from a wearable medical sensor and preprocessing the received data;
receiving data from previous missions from a exemplar-replay-style continuous learning (CL) algorithm;
analyzing the data for the current mission to determine if more output neurons and/or detection heads are needed in a multi-headed deep neural network (DNN) model for the current mission, and if so, expanding the DNN model include more output neurons and/or detection heads;
train the DNN model jointly with substantially equal data from the current mission and previous missions; and
preserving or generating data by either:
obtaining the average training loss of each data instance in the current mission after training, computing a threshold value for data preservation, and preserving data instances whose average training loss values are above the threshold value for future replays; or
modeling a joint multivariate probability distributions of real training data through both parametric and non-parametric density estimation methods, and sampling as much synthetic data as required from the joint multivariate probability distributions.
